(19) United States
(12) Patent Application Publication
(10) Pub. No.: US 2010/0048717 A1
(43) Pub. Date: Feb. 25, 2010

(54) CATHEPSIN B INHIBITORS

(75) Inventors: Jacques Yves Gauthier, Laval (CA); Vouy-Linh Truong, Pierrefonds (CA)

Correspondence Address: MERCK AND CO., INC, P O BOX 2000, RAHWAY, NJ, 07065-0907, US

(73) Assignee: MERCK FROSST CANADA LTD, Merk & Co., Inc., Rahway, NJ (US)

(21) Appl. No.: 12/442,757

(22) PCT Filed: Sep. 24, 2007
(86) PCT No.: PCT/CA07/01715

§ 371 Date: Mar. 25, 2009

Related U.S. Application Data
(60) Provisional application No. 60/847,025, filed on Sep. 25, 2006.

(51) Int. Cl. A61K 31/277; C07C 255/46; A61P 43/00

(52) U.S. Cl. 514/618; 558/393

(57) ABSTRACT

Compounds of formula (I): are found to be selective inhibitors of cathepsin B, and hence useful in treating a variety of pathological conditions.

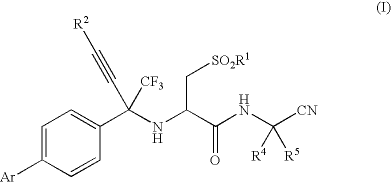

This invention relates to compounds for use in therapeutic treatment of the human body. In particular, there is provided a class of novel compounds which are selective inhibitors of cathepsin B, and hence suitable for use in treating a variety of diseases which are mediated by cathepsin B.

The cathepsins are a family of cysteine proteases belonging to the papain superfamily. Cysteine proteases function in the normal physiological as well as pathological degradation of connective tissue. Aberrant activity of cysteine proteases, e.g. as a result of increased expression or enhanced activation, may have pathological consequences, and cysteine proteases have been associated with numerous disease states such as arthritis, muscular dystrophy, inflammation, tumor invasion, glomerulonephritis, malaria, periodontal disease and others. Cathepsins play a major role in intracellular protein degradation, turnover and remodelling, and at least 8 distinct cathepsins are known (identified as cathepsins B, F, H, L, K, S, W and Z). Increased levels of cathepsin B and redistribution of the enzyme are found in tumours, suggesting a role for cathepsin B in tumor invasion and metastasis. In addition, aberrant cathepsin B activity is implicated in rheumatoid arthritis, osteoarthritis, pneumocystis carinii, acute pancreatitis, inflammatory airway disease and bone and joint disorders. Inhibitors of cathepsin B and/or cathepsin S have been recommended for use in treating chronic obstructive pulmonary disease (COPD) (WO 2004/089395).

Furthermore, recent studies suggest that cathepsin B plays a pivotal role in Alzheimer's disease and other dementing conditions.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. Variability in the site of the proteolysis mediated by y-secretase results in Aβ of varying chain length, e.g. Aβ(1-38), Aβ(1-40) and Aβ(1-42). N-terminal truncations such as Aβ(4-42) are also found in the brain, possibly as a result of variability in the site of proteolysis mediated by β-secretase. For the sake of convenience, expressions such as “AP(1-40)” and “Aβ(1-42)” as used herein are inclusive of such N-terminal truncated variants. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Other dementing conditions associated with deposition of Aβ in the brain include cerebral amyloid angiopathy, hereditary cerebral haemorrhage with amyloidosis, Dutch-type (HCHWA-D), multi-infarct dementia, dementia pugilistica and Down syndrome.

Hook et. Al. (J. Neurochem., 2002, 81, 237-56) identified two distinct pathways leading to secretion of Aβ, namely a regulated secretory pathway and a constitutive secretory pathway, and showed that different β-secretase enzymes were involved in these distinct pathways. Later work by the same group (Hook et. al., Biol. Chem., 2005, 386, 931-40) showed that cathepsin B acts as β-secretase in the regulated pathway, which is the major source of secreted extracellular Aβ. Hence, inhibitors of cathepsin B, in particular selective inhibitors, are of great interest as a potential treatment of AD.

WO 2005/028429 discloses a class of compounds active against cathepsins B, K, L, F and/or S, but does not disclose the compounds of the present invention, and does not disclose selective inhibition of cathepsin B.

According to the invention there is provided a compound of formula I:

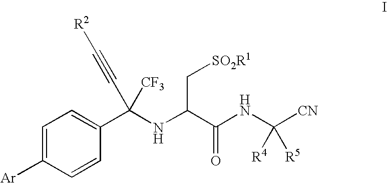

or a pharmaceutically acceptable salt or hydrate thereof; wherein:

R¹ represents C₁₋₆alkyl, C₁₋₆ haloalkyl, C₃₋₆cycloalkyl, C₃₋₆cycloalkylC₁₋₆alkyl, aryl or arylC₁₋₆alkyl, wherein cycloalkyl is optionally substituted with C₁₋₃haloalkyl, and wherein aryl is optionally substituted with 1 to 3 substituents independently selected from C₁₋₆alkyl, halo, C₁₋₆ haloalkyl, C₃₋₆cycloalkyl, C₁₋₆haloalkoxy, —SR^{a}, —S(O)R^{a}, —S(O)₂R^{a}, —OR^{a}, NR^{b}R^{c} and cyano;

R² represents H, C₁₋₆alkyl, C₁₋₆ haloalkyl, C₃₋₆cycloalkyl, C₃₋₆cycloalkylC₁₋₆alkyl or aryl, wherein said aryl is optionally substituted with 1 to 3 substituents independently selected from C₁₋₆alkyl, CH(OH)C₁₋₆alkyl, C₂₋₆alkenyl, halo, C₁₋₆haloalkyl, CH(OH)C₁₋₆haloalkyl, C₃₋₆ cycloalkyl, C₁₋₆haloalkoxy, —SR^{a}, —S(O)R^{a}, —S(O)₂R^{a}, —S(O)₂NR^{b}R^{c}, —OR^{a}, NR^{b}R^{c}, cyano, —C(O)OR^{a}, —C(O)R^{a}, and —C(O)NR^{b}R^{c};

Ar represents aryl or heteroaryl either of which optionally bears up to 3 substituents independently selected from halogen, CN, R³, OR³, COR³, CO₂R³, SR³, S(O)R³ and SO₂R³;

R³ represents C₁₋₆alkyl, C₁₋₆haloalkyl, C₂₋₆alkenyl or C₃₋₆cycloalkyl, any of which optionally bears an OH substituent;

R⁴ and R⁵ independently represent H, C₁₋₆alkyl or C₂₋₆alkenyl, or R⁴ and R⁵ together with the carbon atoms to which they are attached complete a C₃₋₆cycloalkyl ring;

R^{a} is hydrogen or C₁₋₆alkyl;

R^{b} and R^{c} are independently hydrogen or C₁₋₆alkyl; or R^{b} and R^{c}, when attached to a nitrogen atom, together complete a 4- to 6-membered ring optionally having a second heteroatom selected from O, S and N—R^{d}; and

R^{d} is hydrogen or C₁₋₆alkyl.

Where a variable occurs more than once in formula I, the identity taken by said variable at any particular occurrence is independent of its identity at any other occurrence.

Unless otherwise stated, the following terms have the meanings indicated below:

“Alkyl” as well as other groups having the prefix “alk” such as, for example, alkoxy, alkanoyl, and the like, means carbon chains which may be linear or branched or combinations thereof. Examples of alkyl groups include methyl, ethyl, propyl, isopropyl, butyl, sec- and tert-butyl, pentyl, hexyl and the like.

“Alkenyl” means carbon chains which may be linear or branched or combinations thereof containing at least 1 carbon to carbon double bond. Examples of alkenyl groups include ethenyl, 1-propenyl, 2-propenyl, 2-methyl-1-propenyl, 1-butenyl and 1-hexenyl.

“Aryl” means any stable monocyclic or bicyclic carbon ring of up to 10 atoms wherein at least one ring is aromatic carbocycle. In cases where the aryl substituent is bicyclic and the second ring is non-aromatic (e.g., cycloalkyl, cycloalkenyl, heterocyclyl), it is understood that attachment is via the aromatic ring. Examples of aryl group include phenyl, naphthyl, tetrahydronaphthyl, methylenedioxyphenyl, 1,2,3,4-tetrahydroquinolin-5-yl, 4-or 5-indanyl, and 4- or 5-indenyl.

“Cycloalkyl” means carbocycles containing no heteroatoms, and includes mono- and bicyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. In cases where the cycloalkyl substituent is bicyclic and the second ring is aryl, heteroaryl or heterocyclyl, it is understood that attachment is via the non-aromatic carbocyclic ring. Examples of cycloalkyl include cyclopropyl, cyclobutyl, cyclopentyl, cyclohexyl, decahydronaphthalene, adamantane, indanyl, indenyl, 1,2,3,4-tetrahydronaphthalene and the like.

“Haloalkyl” means an alkyl radical as defined above wherein at least one and up to all of the hydrogen atoms are replaced with a halogen. Examples of such haloalkyl radicals include chloromethyl, 1-bromoethyl, fluoromethyl, difluoromethyl, trifluoromethyl, 2,2,2-trifluoroethyl and the like.

“Halogen” or “halo” means fluorine, chlorine, bromine or iodine.

“Heteroaryl” means a stable monocyclic or bicyclic ring of up to 10 atoms wherein at least one ring is aromatic and contains from 1 to 4 heteroatoms selected from the group consisting of O, N and S. Heteroaryl groups within the scope of this definition include, but are not limited to, pyrrolyl, imidazolyl, pyrazolyl, pyridyl, pyrimidinyl, pyrazinyl, pyridazinyl, furanyl, thienyl, oxazolyl, isoxazolyl, oxadiazolyl, thiazolyl, isothiazolyl, thiadiazolyl, triazolyl, tetrazolyl, indolyl, isoindolyl, benzimidazolyl, benzofuranyl, benzothienyl, benzofurazanyl, benzopyrazolyl, benzotriazolyl, benzoxazolyl, benzothiazolyl, benzisoxazolyl, benzisothiazolyl, quinolinyl, isoquinolinyl, cinnolinyl, quinazolinyl, quinoxalinyl, indolinyl, indolazinyl, indazolyl, isobenzofuranyl, naphthyridinyl, tetrazolopyridyl, dihydrobenzoimidazolyl, dihydrobenzofuranyl, dihydrobenzothiophenyl, dihydrobenzoxazolyl, dihydroindolyl, dihydroquinolinyl, tetrahydroquinolinyl. In cases where the heteroaryl substituent is bicyclic and one ring is non-aromatic (e.g, cycloalkyl, cycloalkenyl or heterocyclyl), it is understood that attachment is via the heteroaromatic ring; if both rings are aromatic and one contains no heteroatom, the attachment can be via either ring. If the heteroaryl contains nitrogen atoms, it is understood that the corresponding N-oxides thereof are also encompassed by this definition.

Compounds described herein contain at least two asymmetric centers and may thus exist as enantiomers and as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Alternatively, any enantiomer or diastereomer of a compound of the general Formula I may be obtained by stereospecific synthesis using optically pure starting materials or reagents of known configuration.

In a particular embodiment the compound of formula I has the stereochemistry shown in formula I(a):

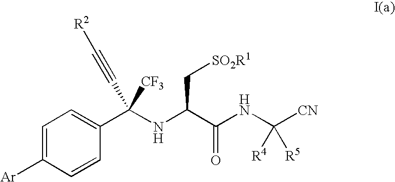

Some of the compounds described herein contain one or more olefinic double bonds which may give rise to E and Z geometric isomers. Unless specified otherwise, both forms are encompassed by the invention.

Some of the compounds described herein may exist as isomers having different points of attachment of hydrogen, referred to as tautomers. An example of such is a ketone and its enol form known as keto-enol tautomers. The individual tautomers as well as mixtures thereof are encompassed by the invention.

The term “pharmaceutically acceptable salt” refers to salts prepared from pharmaceutically acceptable non-toxic bases or acids. When the compound of the present invention is acidic, suitable salts can be conveniently prepared by neutralization with pharmaceutically acceptable non-toxic inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Pharmaceutically acceptable organic non-toxic bases from which salts can be formed include arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, suitable salts can be conveniently prepared by neutralisation with pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

In a subset of the compounds of formula I R¹ represents C₁₋₆alkyl (such as methyl, ethyl, n-propyl, isopropyl or t-butyl), C₁₋₆ haloalkyl (such as CF₃ or CH₂CF₃), C₃₋₆cycloalkyl (such as cyclopropyl, cyclobutyl, cyclopentyl or cyclohexyl), C₃₋₆cycloalkylC₁₋₆alkyl (such as cyclopropylmethyl), phenyl or benzyl, said phenyl or benzyl being optionally substituted with 1 to 3 substituents independently selected from C₁₋₆alkyl, halo, C₁₋₆ haloalkyl, C₁₋₆ haloalkoxy, —SR^{a}, —S(O)₂R^{a}, —OR^{a}, NR^{b}R^{c} and cyano. If more than one substituent is present on said phenyl or benzyl, preferably not more than one of said substituents is other than C₁₋₆alkyl, halo, C₁₋₆haloalkyl or C₁₋₆haloalkoxy. In one embodiment R¹ represents C₁₋₆alkyl or C₃₋₆cycloalkyl, and in a particular embodiment R¹ represents methyl.

In another subset of the compounds of formula I R² represents H, C₁₋₆alkyl, C₁₋₆ haloalkyl or C₃₋₆cycloalkyl. In a particular embodiment R² represents C₃₋₆cycloalkyl, most suitably cyclopropyl.

In another subset of the compounds of formula I Ar represents phenyl or 5- or 6-membered heteroaryl, any of which optionally bears up to 3 substituents independently selected from halogen, CN, R³, OR³, COR³, CO₂R³, SR³, S(O)R³ and SO₂R³. Very suitably, Ar represents phenyl or 6-membered heteroaryl (such as pyridyl), optionally substituted as described previously. In a particular embodiment Ar represents phenyl which is substituted in the 4-position. Preferred substituents include R³, S(O)R³and SO₂R³ where R³ is as defined previously. Specific examples of groups represented by Ar include phenyl substituted in the 4-position with CH(OH)CHF₂ and phenyl substituted in the 4-position with S(O)Me or SO₂Me.

In another subset of the compounds of formula I R⁴ and R⁵ are independently selected from H, C₁₋₆alkyl (such as methyl, ethyl or propyl) and C₂-₆alkenyl (such as allyl). In an alternative subset, R⁴ and R⁵ complete a C₃₋₆cycloalkyl group such as cyclopropyl, cyclobutyl, cyclopentyl or cyclohexyl. In a particular embodiment R⁴ and R⁵ complete a cyclopropyl ring.

Compounds of formula I may be obtained by coupling of a boronic acid derivative Ar—B(OR)₂ with an aryl halide (1):

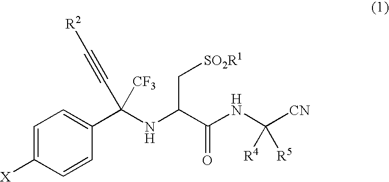

where R represents H or the two R groups complete a cyclic boronate ester such as pinacolate, X represents Cl, Br or 1, and R¹, R², R⁴ and R⁵ have the same meanings as before. The coupling takes place under standard Suzuki conditions, e.g. in DMF in the presence of alkali metal carbonate and a Pd catalyst such as (diphenylphosphinoferrocene)Pd(II)Cl₂ with heating at about 80° C.

Compounds (1) are available by coupling of CN—C(R⁴)(R⁵)-NH₂ with an acid of formula (2):

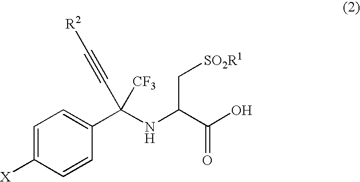

where X, R¹, R², R⁴ and R⁵ have the same meanings as before. Any of the standard procedures for amide coupling may be followed, e.g. use of a peptide coupling reagent such as HATU or EDC and a tertiary amine such as DIPEA or TEA in a solvent such as DM F at 0° C.

The acids (2) are available from oxidation of alcohols (3):

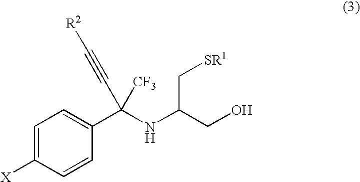

where X, R¹ and R² have the same meanings as before. The oxidation is preferably carried out in two stages, with a first step involving oxidation of the thioether group by treatment with persulfate, and a second step involving oxidation of the primary alcohol group e.g. by treatment with periodic acid and chromium trioxide.

Alcohols (3) are obtainable by ring opening of oxazolidines (4) with R²—C═C—Li:

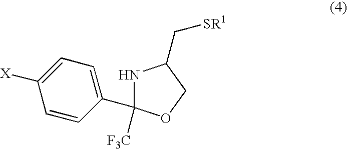

where X, R¹ and R² have the same meanings as before. The reaction may be carried out at −78° C. to −5° C. under anhydrous conditions in THF.

Oxazolidines (4) are obtainable by condensation of X-C6H₄COCF₃ with an amino alcohol (5):

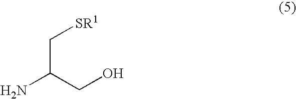

where X and R¹ have the same meanings as before. The reaction takes place under dehydrating conditions, e.g. in refluxing toluene in the presence of pyridinium tosylate with azeotropic removal of water.

Amino alcohols (5) are obtainable by S-alkylation of cysteine alkyl ester followed by borohydride reduction of the ester group.

The starting materials and reagents used in the schemes described above are either commercially available or available by routine chemical modification of commercial materials.

Compounds according to the invention exist as optical isomers due to the presence of two or more chiral centres. Such compounds may be prepared in racemic form, or individual enantiomers may be prepared either by enantiospecific synthesis or by resolution. The novel compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallisation and regeneration of the free base. The novel compounds may also be resolved by formation of diastereomeric esters or amides, followed by chromatographic separation and removal of the chiral auxiliary. Alternatively, racemic intermediates in the preparation of compounds of formula I may be resolved by the aforementioned techniques, and the desired enantiomer used in subsequent steps.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} ed., 1999. The protecting groups may be removed at a convenient subsequent stage using methods known from the art.

The compounds of the invention have the useful property of selectively inhibiting cathepsin B. In particular, the compounds show selectivity for cathepsin B over cathepsin S and cathepsin L. The compounds are therefore useful in treatment or prevention of cathepsin B dependent diseases and conditions in mammals, especially humans.

Therefore, in another aspect the present invention provides a method for the prevention or treatment of cathepsin B dependent conditions in a mammal which comprises administering to said mammal a therapeutically effective amount of a compound of formula I or a pharmaceutically acceptable salt or hydrate thereof. This aspect also encompasses the use of a compound of formula I or a pharmaceutically acceptable salt or hydrate thereof for the manufacture of a medicament for the prevention or treatment of cathepsin B dependent conditions in a mammal. Examples of cathepsin B dependent conditions include tumor invasion and metastasis, rheumatoid arthritis, osteoarthritis, pneumocystis carinii, acute pancreatitis, inflammatory airway disease, COPD, bone and joint disorders, and diseases associated with deposition of β-amyloid in the brain.

According to a further aspect of the invention there is provided the use of a compound according to formula I as defined above, or a pharmaceutically acceptable salt or hydrate thereof, for the manufacture of a medicament for treatment or prevention of a disease associated with the deposition of β-amyloid in the brain.

The disease associated with deposition of AP in the brain is typically Alzheimer's disease (AD), cerebral amyloid angiopathy, HCHWA-D, multi-infarct dementia, dementia pugilistica or Down syndrome, preferably AD.

In a further aspect, the invention provides the use of a compound of Formula I as defined above, or a pharmaceutically acceptable salt or hydrate thereof, in the manufacture of a medicament for treating, preventing or delaying the onset of dementia associated with Alzheimer's disease, cerebral amyloid angiopathy, HCHWA-D, multi-infarct dementia, dementia pugilistica or Down syndrome.

The invention also provides a method of treating or preventing a disease associated with deposition of Aβ in the brain comprising administering to a patient in need thereof a therapeutically effective amount of a compound of Formula I as defined above or a pharmaceutically acceptable salt or hydrate thereof.

In a further aspect, the invention provides a method of treating, preventing or delaying the onset of dementia associated with Alzheimer's disease, cerebral amyloid angiopathy, HCHWA-D, multi-infarct dementia, dementia pugilistica or Down syndrome comprising administering to a patient in need thereof a therapeutically effective amount of a compound of Formula I as defined above or a pharmaceutically acceptable salt or hydrate thereof.

In a further aspect, the invention provides a method for attenuating the secretion of P-amyloid from a mammalian cell comprising contacting said cell with an effective amount of a compound of formula I or a pharmaceutically acceptable salt or hydrate thereof.

In one embodiment of the invention, the compound of Formula I is administered to a patient suffering from AD, cerebral amyloid angiopathy, HCHWA-D, multi-infarct dementia, dementia pugilistica or Down syndrome, preferably AD.

In an alternative embodiment of the invention, the compound of Formula I is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the compound of Formula I is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the compound of Formula I is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42),

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

The compounds of Formula I are typically used in the form of pharmaceutical compositions comprising one or more compounds of Formula I and a pharmaceutically acceptable carrier. Accordingly, in a further aspect the invention provides a pharmaceutical composition comprising a compound of formula I as defined above, or a pharmaceutically acceptable salt or hydrate thereof, and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

For treating or preventing a cathepsin B dependent condition such as Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of I to 4 times per day. In some cases, however, a dosage outside these limits may be used.

The compounds of Formula I optionally may be administered in combination with one or more additional compounds known to be useful in the treatment or prevention of diseases or conditions for which compounds of formula I are useful. In the case of AD, such additional compounds include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which inhibit or modulate the secretion of Aβ (including γ-secretase inhibitors, γ-secretase modulators, and GSK-3α inhibitors), compounds which inhibit the aggregation of Aβ, and antibodies which selectively bind to Aβ. Such additional compounds also include growth hormone secretagogues, as disclosed in WO 2004/110443.

In this embodiment of the invention, the amyloid modifier may be a compound which inhibits the secretion of Aβ, for example an inhibitor of γ-secretase (such as those disclosed in WO 01 /90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621, and also including GSK-3 inhibitors, particularly GSK-3(x inhibitors, such as lithium, as disclosed in Phiel et al, Nature, 423 (2003), 435-9.

Within this embodiment, the amytoid modifier is advantageously a γ-secretase inhibitor, preferred examples of which include a compound of formula XI:

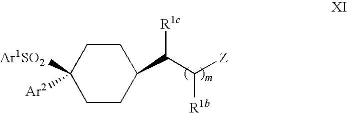

wherein m, Z, R^{1b}, R^{1c}, Ar¹ and Ar² are as defined in WO 03/018543;

or a pharmaceutically acceptable salt thereof

Such compounds may be prepared as described in WO 03/018543. Preferred examples include those defined by formula XIa:

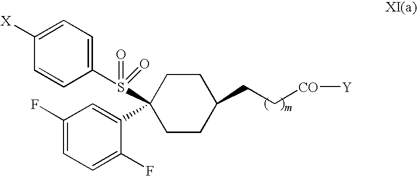

and the pharmaceutically acceptable salts thereof, wherein m is 0 or 1, X is Cl or CF₃, and Y is OH, OC₁₋₆alkyl, NH₂ or NHC₁₋₆alkyl. Particular examples include those in which m is 1 and Y is OH (or the sodium salts thereof), and those in which m is 0 and Y is NH₂ or NHC₁₋₆alkyl.

Another preferred class of γ-secretase inhibitors for use in this embodiment of the invention is that defined by formula XII:

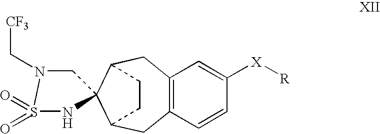

wherein X and R are as defined in WO 03/093252;
or a pharmaceutically acceptable salt thereof.

X is very aptly 5-substituted-thiazol-2-yl, 5-substituted-4-methylthiazol-2-yl, 5-substituted-1-methylpyrazol-3-yl, 1-substituted-imidazol-4-yl or 1-substituted-1,2,4-triazol-3-yl. Preferably, R represents optionally-substituted phenyl or heteroaryl such as phenyl, monohalophenyl, dihalophenyl, trihalophenyl, cyanophenyl, methylphenyl, methoxyphenyl, trifluoromethylphenyl, trifluoromethoxyphenyl, pyridyl, monohalopyridyl and trifluoromethylpyridyl, wherein “halo” refers to fluoro or chloro. Particularly preferred identities of R—X— include 5-(4-fluorophenyl)-1-methylpyrazol-3-yl, 5-(4-chlorophenyl)-1-methylpyrazol-3-yl and 1-(4-fluorophenyl)imidazol-4-yl. Such compounds may be prepared by methods disclosed in WO 03/093252.

As opposed to an inhibitor of γ-secretase, the amyloid modifier may be a modulator of γ-secretase which selectively attenuates the production of Aβ(1-42). This results in preferential secretion of the shorter chain isoforms of Aβ, which are believed to have a reduced propensity for self-aggregation and plaque formation, and hence are more easily cleared from the brain, and/or are less neurotoxic. Compounds showing this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70). Compounds which modulate the activity of PPARα and/or PPARδ are also reported to have the effect of lowering Aβ(1-42) (WO 02/100836). US 2002/0015941 teaches that agents which potentiate capacitative calcium entry activity can lower Aβ(1-42). Further classes of compounds capable of selectively attenuating Aβ(1-42) production are disclosed on WO 2005/054193, WO 2005/013985, WO 2006/008558, WO 2005/108362 and WO 2006/043064.

Alternatively, the amyloid modifier may be a compound which inhibits the aggregation of Aβ or otherwise attenuates is neurotoxicicity. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 (in particular 3-aminopropane-1-sulfonic acid, also known as tramiprosate or Alzhemed™); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191. Further examples include phytic acid derivatives as disclosed in U.S. Pat. No. 4,847,082 and inositol derivatives as taught in US 2004/0204387.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble AP from biological fluids, as described in WO 03/016466, WO 03/016467, WO 03/015691 and WO 01/62801. Suitable antibodies include humanized antibody 266 (described in WO 01/62801) and the modified version thereof described in WO 03/016466.

As used herein, the expression “in combination with” requires that therapeutically effective amounts of both the compound of Formula I and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the compound of Formula I.

### EXAMPLES

### Biological Activity—In Vitro Assays

Enzyme activity assays: Assays of Cat S were carried out in 50 mM MES pH 6.5, 100 mM NaCl, 2.5 mM DTT, 2.5 mM EDTA, 0.001% w/v BSA, 10% DMSO and 40 μM Z-Val-Val-Arg-AMC as substrate. Assays of Cat B were carried out in 50 mM MES pH 6.0,2.5 mM DTT, 2.5 mM EDTA, 0.001% Tween-20, 10 % DMSO and 83 ,M Boc-Leu-Lys-Arg-AMC as substrate. Assays of humanized rabbit Cat K and Cat L were carried out in 50 mM MES pH 5.5, 2.5 mM DTT, 2.5 mM EDTA, 10% DMSO and 2 μM Z-Leu-Arg-AMC as substrate. Prior to the addition of substrate, inhibitor (10.0 μM to 0.02 nM) was pre-incubated for 15 min with each enzyme (0.1-1 nM) to allow the establishment of the enzyme-inhibitor complex. Substrate was then added and the enzyme activity measured from the increase of fluorescence at 460 nm (λₑₓ=355 nm). Assays were performed in 96-well plate format and the plate read using a Gemini EM (Molecular Devices) plate reader. The substrate concentrations employed represent Kₘ or sub-Kₘ values. The percent inhibition of the reaction was calculated from a control reaction containing only vehicle. IC₅₀ curves were generated by fitting percent inhibition values to a four parameter logistic model (SoftmaxPro, Molecular Devices). Compounds of formula (I) generally have IC50 values of about 1 μM or lower; more typically they have IC50 values of about 50 nM or lower against cathepsin B. Compounds exemplified herein were found to be >75-fold selective for rat cathepsin B over rat cathepsin S and >400-fold selective for rat cathepsin B over rat cathepsin L.

### Abbreviations Used

The following abbreviations have the meanings indicated, unless stated otherwise in the specification: ACN=acetonitrile; DIPEA=N,N-diisopropylethylamine; DMF=dimethylformamide; EDC=N-Ethyl-N′-(3-dimethylaminopropyl)carbodiimide; eq.=equivalent(s); ES (or ESI)—MS=electron spray ionization—mass spectroscopy; Et=ethyl; EtOAc=ethyl acetate; HATU=O-(7-azabenzotriazol-1-yl)-N,N,N′,N′-tetramethyluronium hexafluorophosphate; MTBE=methyl t-butyl ether; MeOH=methanol; MHz=megahertz; NMR=nuclear magnetic resonance; PPTS=p-Toluenesulfonic acid pyridine salt; RT=Room temperature; TEA=triethylamine; THF=tetrahydrofuran; Ts=toluenesulfonyl.

### Example 1

### N-(1-cyanocyclopropyl)-N²-[(1R)-3-cyclopropyl-1-{4′-[(1R)-2,2-difluoro-1-hydroxyethyl]biphenyl-4-yl}-1-(trifluoromethyl)prop-2-yn-1-yl]-3-(methylsulfonyl)-L-alaninamide

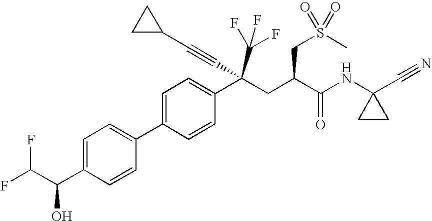 [0077] Step 1. (2R,4R)-2-(4-bromophenyl)-4-[(methylthio)methyl]-2-(trifluoromethyl)-1,3-oxazolidine. 10N sodium hydroxide (6.98 mL) was added to a 0° C. mixture of (2R)-2-amino-3-(methylthio)-propan-1-ol hydrochloride (11 g, 69.8 mmol) and toluene (233 mL) and the mixture was stirred for 30 min. 2,2,2-trifluoro-1-(4-bromophenyl)ethanone (15.9 g, 62.8 mmol) and PPTS (1.061 g, 5.5 mmol) were added and the mixture was heated to reflux with continuous water removal (Dean-Stark apparatus) for 36 hours. The mixture was cooled, stripped to dryness and purified by silica gel chromatography (1:10 ethyl acetate/hexanes) to provide 18.8 g of the (S,R) and the (R,R) isomers as a 1.5:1 mixture.
Step 2. (2R)-2-{[(1R)-1-(4-bromophenyl)-3-cyclopropyl-1-(trifluoromethyl)prop-2-yn-1-yl]amino}-3-(methylthio)propan-1-ol. To −35° C. solution of cyclopropylacetylene (211 mmol, 4.5 eq; 25 mL of Aldrich reagent) in tetrahydrofuran (350 mL) was added n-butyllithium 2M in hexanes (94 mL, 180 mmol, 4 eq). The mixture was stirred at −35° C. for 30 minutes and then warmed to −5° C. for 30 min. It was cooled again to −78° C. and the intermediate from Step 1 (16.7 g, 46.9 mmol) in tetrahydrofuran (50 mL) was added slowly at −78° C. The mixture was reacted for 2 hrs at −78° C. and then warmed up to −5° C. After ˜0.5 hr at −5° C., the mixture turned brown-red and was immediately cooled down and quenched by pouring slowly into water, ice and MTBE. The pH was adjusted to ˜3 and the mixture stirred 0.5 hr. It was extracted twice with MTBE. The combined organic layers were washed with brine, dried with magnesium sulfate and the solvent was removed in vacuo to give 18.2 g. of material. This material was purified by chromatography on silica gel using 1:4 EA:H to yield 4.7 g. of impure product (19-F shows trace of isomer) which was used as such in the next step.
Step 3. (2R)-2-{[(1R)-1-(4-bromophenyl)-3-cyclopropyl-1-(trifluoromethyl)prop-2-yn-1-yl]amino}-3-(methylsulfonyl)propan-1-ol. To a21° C. solution of the sulphide from Step 2 (1.4 g, 3.32 mmol) in acetone (30 mL) was added a solution of oxone monopersulfate (6.12 g, 9.96 mmol, 3 eq) in 2 mL of water. The biphasic reaction mixture was stirred at 21° C. for 2 h. Acetone was removed in vacuo and ethyl acetate was added to the residue. It was washed with an icy solution of Na2S2O3, with brine and the organic layer was dried with MgSO4. Concentration under vacuum afforded 1.5 g. of the title compound used as such in the next step.
Step 4. N-[(1R)-1-(4-bromophenyl)-3-cyclopropyl-1-(trifluoromethyl)prop-2-yn-1-yl]-3-(methylsulfonyl)-L-alanine. To a solution of the alcohol from Step 3 (1.4 g, 3.08 mmol) in acetonitrile (15 mL) at 0° C. was added dropwise a freshly prepared solution (28 mL, 12.32 mmol, 4 eq) of periodic acid/CrO3 [prepared as in Zhao M. et al. Tet. Lett. (1998), 39, 5323-5326; 5.7 g of periodic acid and 12 mg of CrO3 dissolved in 57 mL of 0.75% V/V water/ACN]. The reaction mixture was stirred at 0° C. for 3 h and then poured into an icy aqueous Na2HPO4 solution. The pH was adjusted to 3 with 1N HCl and the mixture was extracted with ethyl acetate. The organic layer was washed with a mixture of saturated brine and water (1:1), followed by an aqueous solution of NaHSO3 and finally with brine. The organic layer was dried with MgSO4 and concentrated under vacuum to afford 1.2 g of the acid used as such in the next step.
Step 4. N²-[(1R)-1-(4-bromophenyl)-3-cyclopropyl-1-(trifluoromethyl)prop-2-yn-1-yl]-N-(1-cyanocyclopropyl)-3-(methylsulfonyl)-L-alaninamide. To a solution of the acid from Step 3 (1.2 g, 2.56 mmol) and 1-cyanocyclopropanaminium chloride (364 mg, 3.07 mmol, 1.2 eq) in N,N-dimethylformamide (5 mL) at 0° C. were added HATU (1.46 g, 3.84 mmol, 1.5 eq) and N,N-diisopropylethylamine (2.3 mL, 13.17 mmol, 5.14 eq). The reaction mixture was stirred at 21° C. overnight and then poured into an icy saturated NaHCO3 solution. It was extracted with ethyl acetate (2×50 mL) and the combined organic layers were washed with a saturated NH4Cl solution and brine. It was dried with MgSO4 and concentrated under vacuum. The residue was purified by chromatography on silica gel (EtOAc/Hexane, 15:85 to 35:65) followed by triturating in MTBE/hexanes to afford the title product (400 mg). 19F-NMR showed only one diastereoisomer.

Title compound: 1H NMR (d₆-acetone, 500 MHz) δ 8.5 (1H, bs), 7.75 (2H, mn), 7.65 (2H, m), 3.95-4.05 (1H, m), 3.55-3.75 (2H, m), 3.3-3.4 (1H, m), 3.1 (3H, s), 1.4-1.6 (3H, m), 1.2-1.3 (3H, m), 0.8-1.0 (3H, m). MS (+ESI): m/z 532.0 and 534.0.
Step 5. N-(1-cyanocyclopropyl)-N²-[(1R)-3-cyclopropyl-1-{4′-[(1R)-2,2-difluoro-hydroxyethyl]biphenyl-4-yl}-1-(trifluoromethyl)prop-2-yn-1-yl]-3-(methylsulfonyl)-L-alaninamide. A mixture of the bromide from Step 4 (0.266 g., 0.5 mmol), (1R)-2,2-difluoro-1-[4-(4,4,5,5-tetramethyl-1,3,2-dioxaborolan-2-yl)phenyl]ethanol (0.17 g.; 0.6 mmol), 2M sodium carbonate (0.75 mL) and DMF (3 mL) was degassed with nitrogen and palladium(II) dichloride (diphenylphosphinoferrocene), 1:1 adduct with dichloromethane (22.8 mgs, 0.028 mmol) was added. The mixture was heated to 75° C. for 4 hrs and then cooled to room temperature. It was poured on dilute aqueous NH4Cl and extracted twice with ethyl acetate. The organic layer was washed with brine and dried with magnesium sulphate. After removal of the solvent, the residue was purified by chromatography on silica using ethyl acetate and hexanes (2:1) to yield the title product (110 mgs; slightly contaminated with pinnacol).

Title compound: MS (+ESI): m/z 610.1/632.1.

### Example 2

### N-(1-cyanocyclopropyl)-N²-[(1R)-3-cyclopropyl-1-[4′-(methylsulfinyl)biphenyl-4-yl]-1-(trifluoromethyl)prop-2-yn-1-yl]-3-(methylsulfonyl)-L-alaninamide

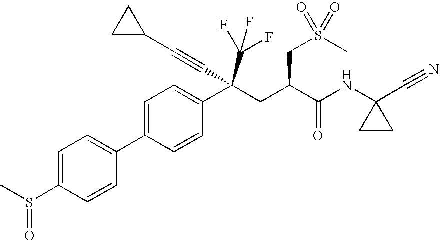 [0086] Step 1. A solution of the bromide from step 4, Example 1 (532 mg, 0.99 mmol), 4,4,5,5-tetramethyl-2-[4-(methylsulfinyl)phenyl]-1,3,2-dioxaborolane (399 mg, 1.5 mmol), 2M sodium carbonate (1.25 mL, 2.5 mmol) and N,N-dimethylformamide (10 mL) were degassed with nitrogen and then palladium(II) dichloride (diphenylphosphinoferrocene), 1:1 adduct with dichloromethane (55 mg, 0.1 mmol) was added. The mixture was heated at 80° C. for 4 hrs. It was cooled and poured on water, NaHCO3 and EA. It was extracted twice with ethyl acetate and the combined organic layers were washed with brine and dried with magnesium sulfate. A portion was purified by chromatography on silica gel using MeOH and CH2C12 (1:25) to yield title compound (166 mgs; contains ˜3% impurities)

Title compound: MS (+ESI): m/z 592.2/614.0.

### Example 3

### N-(1-cyanocyclopropyl)-N²-[(1R)-3-cyclopropyl-1-[4′-(methylsulfonyl)biphenyl-4-yl]-1-(trifluoromethyl)prop-2-yn-1-yl]-3-(methylsulfonyl)-L-alaninamide

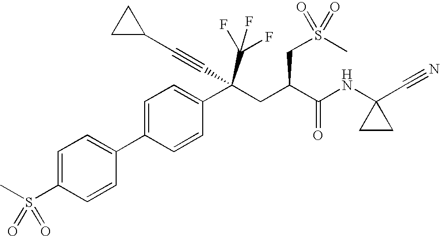

To a −5° C. mixture of the sulfoxide from Example 2 (0.215 g, 0.363 mmol), sodium tungstate dihydrate (5.9 mg, 0.018 mmol), tetrabutylammonium hydrogen sulfate (6.1 mg, 0.018 mmol) in ethyl acetate (20 mL) was added hydrogen peroxide 30% (37.1 uL, 0.363 mmol) and the mixture was stirred at 5° C. for 16 hrs. To the mixture was added dilute NaHSO3 and brine and it was stirred for 10 min. It was extracted twice with ethyl acetate and the combined organic layers were washed with brine and dried with magnesium sulfate. The residue from evaporation was purified by chromatography on silica using ethyl acetate and hexanes (3:1) to yield the title (0.83 g.; contains ˜3% impurities).

Title compound: MS (+ESI): m/z 608.2/630.0.

1. A compound of formula I: 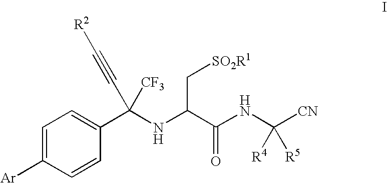
or a pharmaceutically acceptable salt or hydrate thereof; wherein:
R¹ represents C₁₋₆alkyl, C₁₋₆ haloalkyl, C₃₋₆cycloalkyl, C₃₋₆cycloalkylC₁alkyl, aryl or arylC₁₋₆alkyl, wherein cycloalkyl is optionally substituted with C₁₋₃ haloalkyl, and wherein aryl is optionally substituted with 1 to 3 substituents independently selected from C₁₋₆alkyl, halo, C₁₋₆ haloalkyl, C₃₋₆cycloalkyl, C₁₋₆ haloalkoxy, —SR^{a}, —S(O)R^{a}, —S(O)₂R^{a}, —OR^{a}, NR^{b}R^{c} and cyano;
R² represents H, C₁₋₆alkyl, C₁₋₆ haloalkyl, C₃₋₆cycloalkyl, C₃₋₆cycloalkylC₁₋₆alkyl or aryl, wherein said aryl is optionally substituted with 1 to 3 substituents independently selected from C₁₋₆alkyl, CH(OH)C₁₋₆alkyl, C₂₋₆alkenyl, halo, C₁₋₆ haloalkyl, CH(OH)C₁₋₆ haloalkyl, C₃₋₆ cycloalkyl, C₁₋₆haloalkoxy, —SR^{a}, —S(O)R^{a}, —S(O)₂R^{a}, —S(O)₂NR^{b}R^{c}, —OR^{a}, NR^{b}R^{c}, cyano, —C(O)OR^{a}, —C(O)R^{a}, and —C(O)NR^{b}R^{c};
Ar represents phenyl or heteroaryl either of which optionally bears up to 3 substituents independently selected from halogen, CN, R³, OR³, COR³, CO₂R³, SR³, S(O)R³ and SO₂R³;
R³ represents C₁₋₆alkyl, C₁₋₆haloalkyl, C₂₋₆alkenyl or C₃₋₆cycloalkyl, any of which optionally bears an OH substituent;
R⁴ and R⁵ independently represent H, C₁₋₆alkyl or C₂₋₆alkenyl, or R⁴ and R⁵ together with the carbon atoms to which they are attached complete a C₃₋₆cycloalkyl ring;
R^{a} is hydrogen or C₁₋₆alkyl;
R^{b} and R^{c} are independently hydrogen or C₁₋₆alkyl; or R^{b} and R^{c}, when attached to a nitrogen atom, together complete a 4- to 6-membered ring optionally having a second heteroatom selected from O, S and N—R^{d}; and
R^{d} is hydrogen or C₁₋₆alkyl.

2. A compound according to claim 1 having the stereochemistry shown in formula I(a): 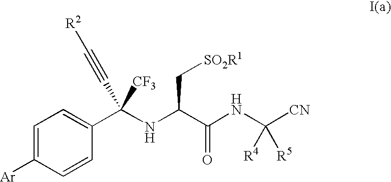
where all variables are as defined in claim 1.

3. A compound according to claim 1 wherein R¹ represents methyl.

4. A compound according to claim 1 wherein R² represents cyclopropyl.

5. A compound according to claim 1 wherein Ar represents optionally substituted phenyl or pyridyl.

6. A compound according to claim 5 wherein Ar represents phenyl which bears a substituent in the 4-position.

7. A compound according to claim 6 wherein the substituent is SO₂CH₃, S(O)CH₃ or CH(OH)CHF₂.

8. A compound according to claim 1 wherein R⁴ and R⁵ complete a cyclopropyl ring.

9. A pharmaceutical composition comprising a compound of formula I as defined in claim 1 or a pharmaceutically acceptable salt or hydrate thereof and a pharmaceutically acceptable carrier.

10. A method for the prevention or treatment of cathepsin B dependent conditions in a mammal which comprises administering to said mammal a therapeutically effective amount of a compound of formula I as defined in claim 1 or a pharmaceutically acceptable salt or hydrate thereof.
